(19) United States
(12) Patent Application Publication
(10) Pub. No.: US 2022/0202906 A1
(43) Pub. Date: Jun. 30, 2022

(54) VASOACTIVE INTESTINAL PEPTIDE (VIP) FOR USE IN THE TREATMENT OF DRUG-INDUCED PNEUMONITIS

(71) Applicant: ADVITA LIFESCIENCE GMBH, Denzlingen (DE)

(72) Inventors: Joachim MÜLLER-QUERNHEIM, Kirchzarten (DE); Björn Christian FRYE, Freiburg (DE)

(21) Appl. No.: 17/595,025

(22) PCT Filed: May 5, 2020
(86) PCT No.: PCT/EP2020/062420

§ 371 Date: Nov. 5, 2021

(30) Foreign Application Priority Data
May 7, 2019 (EP) 19000219.6

(51) Int. Cl. A61K 38/22; C07K 14/575; A61P 11/00

(52) U.S. Cl. CPC A61K 38/2278; A61P 11/00; C07K 14/57563

(57) ABSTRACT

Checkpoint inhibitor-induced pneumonitis (CIP) is characterized clinically by dyspnea, cough and tachypnea. Hypoxia results from a lymphocyte-dominated alveolitis leading to ground glass opacities and consolidations observed by CT scan. Histological findings include lymphocytic infiltrates, granuloma formation and eosinophilic accumulation. In the management of CIP, systemic administration of steroids such as methylprednisolone is the standard therapy. Moreover, CIP in most cases leads to discontinuation of checkpoint inhibitory therapy and steroids limit the therapeutic effect of checkpoint inhibitors resulting in progression of the underlying malignant disease. Therefore, there is a need of other therapeutic options in CIP that ideally could abrogate the alveolar inflammation induced by checkpoint inhibitors without affecting the systemic effect on the immune system. The focus of the present invention is to deliver a solution to that problem by the topic application of VIP (vasoactive intestinal peptide, a peptide of 28 amino acids). A drug for inhalative VIP therapy is commercially available under the name Aviptadil.

The present invention generally relates to Vasoactive Intestinal Peptide (VIP) for use in the treatment of drug-induced pneumonitis. In particular, the present invention relates to VIP for use in the treatment of checkpoint inhibitor related pulmonary pneumonitis (CIP) and methotrexate-induced pneumonitis.

Vasoactive intestinal peptide (VIP) is a 28 amino acid polypeptide. VIP is a neurotransmitter that is extensively distributed in a broad range of tissues and exerts diverse actions on the cardiovascular system, pancreas, digestive tract, respiratory system and urological system. The polypeptide derived its name because of its vasodilating action which modifies the intestinal blood flow. The INN for the vasoactive intestinal peptide (VIP) having 28 amino acids is “Aviptadil”. A pharmaceutical composition for inhalative VIP therapy is commercially available under this name Aviptadil from Advita Lifescience GmbH, Denzlingen, Germany. The VIP is available from Bachem AG, Bubendorf, Switzerland. The amino acid sequence of VIP is available from UniProtKB Database under P01282 (https://www.uniprot.org/uniprot/P01282).

WO 2015/104596 relates to a vasoactive intestinal peptide and its use for switching off and/or preventing harmful and ongoing inflammations in autoimmune and atopic disease.

EP 2 152 741 B1 discloses peptides with improved properties having the biological activity of vasoactive intestinal peptides and their use for the treatment of chronic obstructive pulmonary disease (COPD), cystic fibrosis and allergic lung diseases.

WO 03/061680 teaches the use of compounds having the biological activity of vasoactive intestinal peptide for the treatment of chronic obstructive pulmonary disease.

EP 1 515 745 B1 relates to the use of VIP and VIP-like peptides for the treatment of sarcoidosis. Sarcoidosis is a systemic disease where a triggering factor is not known and that is histologically defined by epitheloid granulomas, the formation of which is not regarded as a general feature of CIP. For sarcoidosis it has been shown that the inhalation of aerosolized VIP leads to a decrease of TNF-release and an increase of regulatory T-cells which results in symptomatic relief (Prasse A. et al.; Inhaled vasoactive intestinal peptide exerts immunoregulatory effects in sarcoidosis; American journal of respiratory and critical care medicine 2010; 182: 540-548).

Interstitial pneumonitis/fibrosis is the most common clinical manifestation associated with drug-induced pulmonary damage. Many chemotherapeutic drugs against cancer can cause interstitial pneumonitis/fibrosis, while several non-cytotoxic drugs have also been implicated. Clinical symptoms usually begin insidiously, progressing over weeks to months with a non-productive cough, exertional dyspnea, fatigue, malaise and weight loss. Bibasilar end-inspiratory rales are commonly observed on examination. There are more acute forms of this syndrome, occurring within hours to days after exposure to the offending agents.

This syndrome of acute pneumonitis is typically associated with nitrosoureas, cyclophosphamide and the mitomycin/vinca alkaloid combination. It has also been described with methotrexate, amiodarone and biologicals. On chest radiography, interstitial pneumonitis frequently manifests as bilateral bibasilar reticular or nodular infiltrates. Pleural effusions are frequently absent but have been described in association with mitomycin, nitrofurantoin, amiodarone and gold salts.

Occasionally, the chest radiograph may be normal, even in the presence of significant symptoms or pulmonary physiological impairment. Patients with interstitial pneumonitis will commonly have a restrictive defect with a reduced diffusion capacity on pulmonary function testing. Diagnosis is often confirmed with bronchoscopy and transbronchial biopsy.

Immune check point inhibitors are an evolving class of drugs used for therapy of different diseases, especially melanoma and non-small cell lung cancer. Their mode of action is a T-cell activation by interfering with coinhibitory pathways of T-cell activation, namely the PD-1 (programmed death-1) receptor and ligands PD-L1 and PD-L2 (programmed death ligands 1 and 2) axis and the CTLA-4 (cytotoxic T-lymphocytes antigen-4) molecule.

CTLA-4 is expressed mainly by T-cells and it competes with the T-cell activating CD28 for its ligands CD80 and CD86. Therefore, CTLA-4 binding to CD80/CD86 leads to a dampened T-cell activation because CD28 lacks its activating ligand(s). CTLA-4 can be targeted by ipilimumab and tremelimumab leading to an exaggerated anti-tumor response.

PD-1 is expressed on T- and B-lymphocytes, natural killer cells and dendritic cells. PD-1 binding by its ligands PD-L1 and PD-L2 leads to a reduced T-cell activation and effector function. Nivolumab and pembrolizumab are monoclonal antibodies targeting PD-1 to enhance immune response against a given malignant tissue.

The T-cell stimulatory effect of anti-CTLA-4 and anti PD-1 antibodies is, however, an unspecific effect leading to a general T-cell activation and thereby propagating autoimmune diseases as side effect of T-cell activation, so-called immune-related adverse events. Immune-related adverse events occur in approximately 10-15% of patients with an incidence of grade 3 of 3-6%. In contrast to immune-related hepatitis or endocrine side effects which are often self-limiting and can be treated symptomatically, checkpoint inhibitor-induced pulmonary manifestations often require high dose steroid therapy. Pulmonary immune-related adverse events (irAEs) occur in approximately 5% of treated patients and exhibit a mortality of 10%.

Checkpoint inhibitor-induced pneumonitis (CIP) is characterized clinically by dyspnea, cough and tachypnea. Hypoxia results from a lymphocyte-dominated alveolitis leading to ground glass opacities and consolidations observed by CT scan. Histological findings include lymphocytic infiltrates and eosinophilic accumulation. Therefore, CIP can be defined as a lymphocyte dominated interstitial illness that is limited to the lung and exhibits mainly a diffuse alveolar damage.

In the management of CIP and other drug-induced pneumonitis, systemic administration of steroids such as methylprednisolone is the standard therapy. Moreover, CIP in most cases leads to discontinuation of checkpoint inhibitory therapy and steroids limit the therapeutic effect of checkpoint inhibitors resulting in progression of the underlying malignant disease.

Therefore, there is a need of other therapeutic options in CIP and other drug-induced pneumonitis that ideally could abrogate the alveolar inflammation induced by checkpoint inhibitors and other drugs without affecting the systemic effect on the immune system. Thus, it is an object of the present invention to provide a solution to that problem embodied by the topic application of VIP.

Though the topic application of steroids (inhaled) is not sufficient to treat CIP and other drug-induced pneumonitis it has surprisingly been observed that the topic treatment with VIP is able to give relief to these patients. This is especially unexpected as the treatment with aerosols is normally regarded to be only efficient if inhaled noxes are the cause of the pulmonary defects. In the context of the present invention, a systemic substance is, however, the cause of drug-induced pneumonitis (such as CIP) and therefore topic administration is not expected to have any great effect. Especially as the aerosol administration of VIP does not lead to an elevated blood level of VIP and therefore does not show a systemic effect.

Therefore, the present invention relates to VIP for use in the treatment of drug-induced pneumonitis, in particular to VIP for use in the treatment of checkpoint inhibitor-induced pneumonitis (CIP) and VIP for use in the treatment of methotrexate-induced pneumonitis.

Preferred embodiments refer to VIP as an active ingredient, together with at least one pharmaceutically acceptable carrier, excipient and/or diluent in a pharmaceutical composition for the use in the treatment of drug-induced pneumonitis.

Such pharmaceutical compositions comprise VIP as an active ingredient, together with at least one pharmaceutically acceptable carrier, excipient, binder, disintegrant, glident, diluent, lubricant, coloring agent, sweetening agent, flavoring agent, preservative or the like. The pharmaceutical compositions suggested to be used according to the present invention can be prepared in a conventional solid or liquid carrier or diluent and a conventional pharmaceutically-made adjuvant at suitable dosage level as is known in the art.

VIP is a peptide which may form pharmaceutically acceptable salts with organic and inorganic acids. Examples of suitable acids for such acid addition salt formation are hydrochloric acid, hydrobromic acid, sulfuric acid, phosphoric acid, acetic acid, citric acid, oxalic acid, malonic acid, salicylic acid, p-aminosalicylic acid, malic acid, fumaric acid, succinic acid, ascorbic acid, maleic acid, sulfonic acid, phosphonic acid, perchloric acid, nitric acid, formic acid, propionic acid, gluconic acid, lactic acid, tartaric acid, hydroxymaleic acid, pyruvic acid, phenylacetic acid, benzoic acid, p-aminobenzoic acid, p-hydroxybenzoic acid, methanesulfonic acid, ethanesulfonic acid, nitrous acid, hydroxyethanesulfonic acid, ethylenesulfonic acid, p-toluenesulfonic acid, naphthylsulfonic acid, sulfanilic acid, camphersulfonic acid, china acid, mandelic acid, o-methylmandelic acid, hydrogen-benzenesulfonic acid, picric acid, adipic acid, D-o-tolyltartaric acid, tartronic acid, a-toluic acid, (o, m, p)-toluic acid, naphthylamine sulfonic acid, and other mineral or carboxylic acids well known to those skilled in the art. The salts are prepared by contacting the free base form with a sufficient amount of the desired acid to produce a salt in the conventional manner.

In another preferred embodiment VIP is provided in a pharmaceutical composition applicable for inhalation.

For inhalation, the pharmaceutical composition is brought in an aerosol form.

In one preferred embodiment of the present invention, the pharmaceutical composition for aerosolization is a liquid. Suitable concentrations of VIP in the liquid pharmaceutical composition range from about 20 μg/ml to 200 μg/ml. Preferably, the liquid pharmaceutical composition comprises VIP from 35 μg/ml to 140 μg/ml composition and particularly preferred from 60 μg/ml to 80 μg/ml composition. Liquids in which the VIP is contained in a salt solution, in particular in a NaCl solution, more particular in a physiological NaCl solution, are preferred.

The aerosol which is used according to the present invention for the treatment of drug-induced pneumonitis preferably comprises droplets, which are small enough to be easily inhaled, and such liquid droplets have a certain diameter which ranges from about 0.5 to about 10 μm, preferably from about 2.0 to about 6.0 μm and especially preferred between about 2.8 and 4.5 μm.

In another preferred embodiment of the present invention, the pharmaceutical composition for aerosolization is a solid pharmaceutical composition and is provided as a powder, wherein the VIP is used in the form of dry particles which have a diameter of about 2.0 to 4.0 μm. Suitable concentrations of VIP in the solid pharmaceutical composition range from about 20 μg/mg to 200 μg/mg. Preferably, the solid pharmaceutical composition comprises VIP from 35 μg/mg to 140 μg/mg composition and particularly preferred from 60 μg/mg to 80 μg/mg composition. Particles in which the VIP is contained in a composition with an inert carrier, in particular with lactose, more particular with lactose-monohydrate (for example InhaLac 230 from Meggle Group GmbH, Wasserburg, Germany) are preferred. The particles my also contain salts such as sodium chloride or sodium phosphates.

Usually, by aerosolization the liquid droplets or dry particles are finely dispersed within a carrier gas. As suitable carrier gas, inert gases such as helium, neon or argon or mixtures thereof can be used. Preferably, however, inert gases which are easily available like nitrogen (N2) or carbon dioxide (CO2) are used. It is also possible to use ambient air, whereby the oxygen content may be reduced.

The characterization of the aerosol regarding droplet or particle diameter and content of VIP can be easily performed by measurement devices known to the person skilled in the art.

In a preferred embodiment, the aerosol is produced by aerosolization of the liquid pharmaceutical composition in an ultrasonic mesh nebulizer. A particularly preferred nebulizer is the M-Neb® dose+ MN-300/8 supplied by Nebu-Tec, Elsenfeld, Germany. Alternatively, however, the aerosol can be produced by commercially available inhalers which meet the requirement of providing an aerosol having the defined size of the droplets. Alternatively, the VIP may be administered in powdered form by a dry powder inhaler or metered dose inhaler, for example the Turbohaler from AstraZeneca.

In one embodiment, aerosolized VIP is administered to a patient in doses ranging from about 140 μg to 560 μg per day. The daily dose may be administered as a single dose, or as multiple doses adding up to the daily dose. Preferably, the daily dose is administered in three to four separate doses. More preferably, the daily dose is given three to four times per day with overnight break. In a preferred embodiment, aerosolized VIP is administered in a dose of 280 μg per day, wherein suitable doses are administered four times per day preferably with overnight break. For example, a daily dose of 280 μg may be administered as four doses of 70 μg per day, followed by an overnight rest period.

The present invention also relates to a corresponding method for the treatment of a patient. Therefore, another object of the present invention is to provide a method for the treatment of a patient with drug-induced pneumonitis, in particular with checkpoint inhibitor induced-pneumonitis (CIP) or methotrexate-induced pneumonitis, comprising administering to the patient Vasoactive Intestinal Peptide (VIP).

In a preferred embodiment, Vasoactive Intestinal Peptide is administered to the patient as an aerosolized pharmaceutical composition by inhalation. Preferably, a liquid pharmaceutical composition is aerosolized for administration. A suitable concentration of Vasoactive Intestinal Peptide in the liquid pharmaceutical composition ranges from 20 μg/ml to 200 μg/ml. Preferably, the concentration of Vasoactive Intestinal Peptide ranges from 35 μg/ml to 140 μg/ml, particularly preferred from 60 μg/ml to 80 μg/ml.

In another preferred embodiment, a powder is aerosolized in order to provide the aerosol for administration. Suitable concentrations of Vasoactive Intestinal Peptide range from 20 μg/mg to 200 μg/mg. Preferably, the concentration of Vasoactive Intestinal Peptide ranges from 35 μg/mg to 140 μg/mg, particularly preferred from 60 μg/mg to 80 μg/mg.

Preferably, a daily dose from 140 μg to 560 μg Vasoactive Intestinal Peptide is administered to the patient.

In one prior art study, for example, patients received 50 μg synthetic VIP (Aviptadil; Bachem, Basel, Switzerland) four times daily by inhalation by way of an ultrasonic nebulizer (Optineb; Nebu-Tec, Elsenfeld, Germany) for 28 days. After advising patients in the details of inhalation, the technical use of the inhalator and the p.i. administration of VIP was feasible for all patients and well tolerated without serious adverse events (cf. EP 1 515 745 B1).

The experiments and studies that have led to the present invention clearly show that the suggested therapy of VIP inhalation does not suffer from severe side effects on the patient's immune system while successfully dampening alveolar inflammation in CIP. This therapy can thus also be used in combination with or even after additional immunosuppressive steroid therapy to reduce or stabilize an alveolar inflammation induced by checkpoint inhibitory therapy.

The present invention is illustrated in more detail in the following examples.

### EXAMPLE 1

Using the M-Neb® dose+ mesh nebulizer MN-300/8 and the respective mouthpiece, VIP has been tested with the COPLEY next generation impactor (NGI). The mass median aerodynamic diameter (MMD) of VIP dissolved in 0.9% NaCl was 3.3-3.5 μm per emitted particle. 85.7% of particles had a diameter <5 μg and the dose delivered at the mouthpiece was 90.2% of the tested dosages.

### EXAMPLE 2

VIP has been tested in 0.9% NaCl solution at different drug concentrations (20 μg/ml, 35 μg/ml, 50 μg/ml, 70 μg/ml, 140 μg/ml, 200 μg/ml, 250 μg/ml, 400 μg/ml). Results show that the respective biological activity is best between 35 μg/ml-140 μg/ml.

### EXAMPLE 3

VIP has been tested in 0.9% NaCl solution at different time points over increasing numbers of breathing cycles. Diseases of the lung parenchyma result in geometric changes in the lung periphery that can minimize the deposition of inhaled particles. The specific breathing by using slow and deep inspiration allows aerosol particles to bypass the upper airways thus making them available for deposition in the lower respiratory tract. The prolonged inspiration allows for suitable settling of aerosols in desired location of the lung. The prolongation of inspiration time and the advanced settling promotes inspiratory deposition before its particles in aerosol can be exhaled. Under these conditions it is possible to have almost 100% of the delivered particles depositing before exhalation begins. Inhalation times between 10 min to 15 min are preferable over short times of inhalation between 2-4 min per treatment because patients can take longer breath cycles.

### EXAMPLE 4

A patient, who was treated with checkpoint inhibitors, developed CIP and steroid treatment led to insufficient control. Because of missing other approved therapeutic options the patient was treated off-label with inhaled VIP therapy initiated at a dose of 4×70 μg/ml per day dosage (280 μg per day with overnight break). With this treatment, the patient's general health ameliorated, his lung function normalized within six months of treatment and the radiological alterations (e.g. consolidations) diminished. Alveolar inflammation as measured by bronchoalveolar lavage was dampened by an increase of regulatory T-cell.

### EXAMPLE 5

A 72 year old female was diagnosed with rheumatoid arthritis according to current guidelines and an immunosuppressive therapy with corticosteroid (15 mg prednisolone/day) and methotrexate (15 mg/week) was started.

Joint involvement improved within one month and steroid dose was tapered. Shortly after finishing steroid dose the patient complained shortness of breath and cough. Lung function demonstrated a restrictive ventilation defect. A CT scan performed demonstrated wide-spread ground glass opacities with an apical predominance.

Bronchoscopy was performed that ruled out an underlying infection (including bacterial culture, PCR for influenza, parainfluenza, human metapneumonia virua, respiratory syncytial virus, pneumocystis jirovecii, tuberculosis). Bronchoalveolar lavage demonstrated a lymphocyte predominance and ex-vivo alveolar lymphocytes demonstrated increased proliferation when cultured with methotrexate.

These findings allow the diagnosis of a methotrexate-induced pneumonitis. Because the patient experienced side effects of previous steroid treatment the patient was treated with inhaled VIP (as depicted in more detail in example 4 above). The inhalation of VIP lead to a clinical amelioration most likely by interfering with the proinflammatory cascade triggered by methotrexate.

1. Vasoactive Intestinal Peptide (VIP) for use in the treatment of drug-induced pneumonitis.

2. Vasoactive Intestinal Peptide for use according to claim 1, wherein the drug-induced pneumonitis is a checkpoint inhibitor-induced pneumonitis.

3. Vasoactive Intestinal Peptide for use according to claim 1, wherein the drug-induced pneumonitis is a methotrexate-induced pneumonitis.

4. Vasoactive Intestinal Peptide for use according to any of claims 1 to 3, wherein it is provided in a pharmaceutical composition applicable for inhalation.

5. Vasoactive Intestinal Peptide for use according to claim 4, wherein the pharmaceutical composition is provided in a liquid form.

6. Vasoactive Intestinal Peptide for use according to claim 5, wherein the concentration of Vasoactive Intestinal Peptide in the pharmaceutical composition is from 20 μg/ml to 200 μg/ml, preferably from 35 μg/ml to 140 μg/ml, particularly preferred from 60 μg/ml to 80 μg/ml.

7. Vasoactive Intestinal Peptide for use according to claim 4, wherein the pharmaceutical composition is provided in a solid form.

8. Vasoactive Intestinal Peptide for use according to claim 7, wherein the concentration of Vasoactive Intestinal Peptide in the pharmaceutical composition is from 20 μg/mg to 200 μg/mg, preferably from 35 μg/mg to 140 μg/mg, particularly preferred from 60 μg/mg to 80 μg/mg.

9. Vasoactive Intestinal Peptide for use according to any of claims 1 to 8, wherein a daily dose ranges from 140 μg to 560 μg Vasoactive Intestinal Peptide.

10. A method for the treatment of patients with drug-induced pneumonitis, comprising administering to the patient Vasoactive Intestinal Peptide (VIP).

11. The method according to claim 10, wherein the drug-induced pneumonitis is a checkpoint inhibitor-induced pneumonitis.

12. The method according to claim 10, wherein the drug-induced pneumonitis is a methotrexate-induced pneumonitis.

13. The method according to any of claims 10 to 12, wherein Vasoactive Intestinal Peptide is administered to the patient as an aerosolized pharmaceutical composition by inhalation.

14. The method according to claim 13, wherein a liquid pharmaceutical composition is aerosolized for administration.

15. The method according to claim 14, wherein the concentration of Vasoactive Intestinal Peptide in the liquid pharmaceutical composition is from 20 μg/ml to 200 μg/ml, preferably from 35 μg/ml to 140 μg/ml, particularly preferred from 60 μg/ml to 80 μg/ml.

16. The method according to claim 13, wherein a solid pharmaceutical composition is aerosolized for administration.

17. The method according to claim 16, wherein the concentration of Vasoactive Intestinal Peptide in the solid pharmaceutical composition is from 20 μg/mg to 200 μg/mg, preferably from 35 μg/mg to 140 μg/mg, particularly preferred from 60 μg/mg to 80 μg/mg.

18. The method according to any of claims 10 to 17, wherein a daily dose from 140 μg to 560 μg Vasoactive Intestinal Peptide is administered.
